(19) United States
(12) Patent Application Publication
(10) Pub. No.: US 2003/0003028 A1
(43) Pub. Date: Jan. 2, 2003

(54) Germicide apparatus including UV lamps and a control system therefor

(76) Inventor: Giancarlo Tomaselli, Milano (IT)

Correspondence Address: James V. Costigan, Esq., Hedman & Costigan, P.C., Suite 2003, 1185 Avenue of the Americas, New York, NY, 10036-2646, US

(21) Appl. No.: 10/177,933

(22) Filed: Jun. 20, 2002

(30) Foreign Application Priority Data
Jul. 2, 2001 (IT) MI2001 A 001405

(51) Int. Cl. A61L 9/20

(52) U.S. Cl. 422/121; 422/120; 250/436; 250/438

(57) ABSTRACT

A germicide apparatus comprises UV lamps and a switching-hour counter devices for indicating an incipient exhaustion condition of the lamps, thereby allowing the apparatus use to timely replace the UV lamps.

### BACKGROUND OF THE INVENTION

The present invention relates to a germicide apparatus, comprising UV lamps and a control system for controlling the UV lamps.

The field of the invention is that of the germicide apparatus, more specifically apparatus including lamps emitting UV radiation at about 254 nm (UVC), having a destructive effect on all the microorganisms which are usually present in air.

These apparatuses can optionally be also provided with a specifically designed ionizing system, of a negative ion type, contributing to the germicide effect of the UVC lamps.

The lamps used in the apparatuses the invention is related to are conventionally used in environments to be held in a sterile condition, such as operating rooms, dentist's surgery rooms, analysis laboratories and the like.

As is known, prior apparatuses for germicide use, including UVC lamps, have the drawback that they cannot control the operation time of the lamps, which time, on the contrary, greatly affects the germicide effect of these apparatuses.

Actually, the UVC lamps have a limited life average time, which usually corresponds to about 6000 hours, after which the germicide efficiency of the lamps starts to decrease.

Moreover, in particular of a closed-loop apparatus, in an exhausted or not efficient condition of the lamps, said apparatus would form a mechanism diffusing microorganisms through the environment, where a forced air circulation would favour a further diffusion of polluting substances.

Furthermore, prior closed-loop apparatuses are also affected by the drawback that they are so designed as to exclusively direct the air jet in a preset direction in the factory, which cannot be modified during the actual use.

Yet another drawback affecting the above mentioned closed loop apparatuses is that the walls of the apparatus casing do not allow to see from the outside the on or off status of the lamps.

### SUMMARY OF THE INVENTION

Accordingly, the aim of the invention is to provide a novel UV germicide apparatus, which also comprises an ionizing system, for indicating in useful time the exhaustion condition of the UVC lamps.

Within the scope of the above mentioned aim, a main object of the present invention is to provide an apparatus of the above mentioned type, affording the possibility of properly orienting the air jet at the outlet of the closed loop germicide apparatus.

Yet another object of the present invention is to provide such a germicide apparatus, which, notwithstanding the provision of the casing usually used in closed loop systems, allows to visually control from the outside the on or off status of the UVC lamps.

According to one aspect of the present invention, the above mentioned aim and objects, as well as yet other objects, which will become more apparent hereinafter, are achieved by a germicide apparatus as claimed in claim 1.

Further features of the inventive apparatus are defined in the remaining claims.

With respect to conventional apparatuses, the inventive apparatus provides the advantage of timely signalling the exhaustion condition of the UVC lamps, starting from their operation time, and calculated as a sum of the time intervals in which the UVC lamps are held in an on condition.

Thus, the user can be timely provided with an indication of the need of replacing the lamps since they would be inefficient on microorganisms, or susceptible to damage the user, as it occurs in closed loop systems including exhausted or poorly operating lamps.

The apparatus according to the invention provides moreover the advantage of orienting the sterilized air jet, so as to cause said air jet to impinge at the desired or preferred place.

Yet another advantage of the inventive apparatus is that it allows to visually control the UVC lamp on or off condition, even in closed-loop system including a tight vessel tightly covering the lamps.

### BRIEF DESCRIPTION OF THE DRAWINGS

The invention will be now disclosed in a more detailed manner with reference to a preferred embodiment thereof which is illustrated, by way of an indicative, but not limitative, example in the accompanying drawings, where:

FIG. 1 is a general perspective view illustrating the apparatus according to the invention;

FIG. 2 illustrates the apparatus of FIG. 1 and the lamps thereof, shown in a broken away manner, as well as the hour counter device including a lamp on and off switching element;

FIG. 3 shows the power supply circuit for power supplying the lamps, in cooperation with the hour counter device shown in FIG. 2;

FIGS. 4 to 6 show the fixing system for fixing the air outlets of the subject apparatus, affording the possibility of variably orienting the sterilized air jet.

### DESCRIPTION OF THE PREFERRED EMBODIMENT

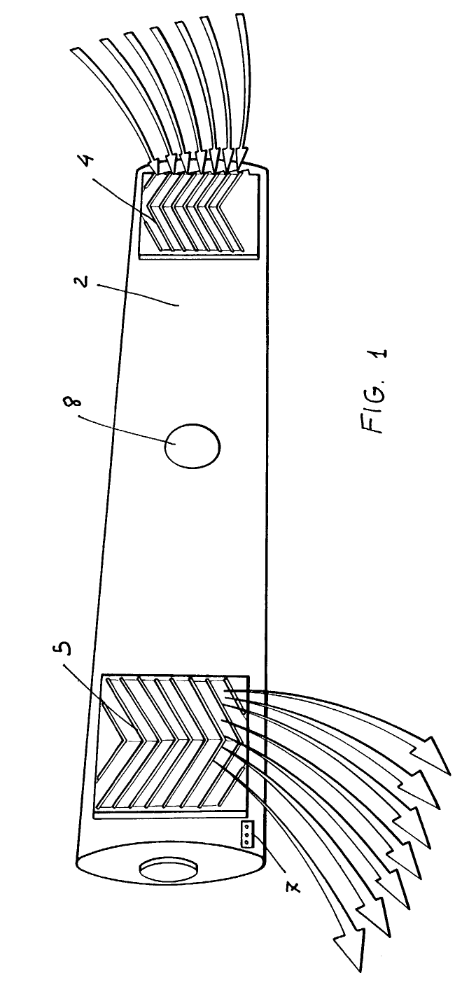

In FIG. 1, the germicide apparatus according to the present invention has been generally indicated by the reference number 1.

In the shown embodiment, the apparatus is of a closed loop type, and does not include any ionizing system.

In this connection it should be pointed out that the invention can also be advantageously applied to other systems, in particular to systems of open loop type, with not covered lamps, either of a direct or of an indirect light type, as well as in systems also including an ionizing device.

The apparatus 1 comprises a casing 2 housing therein a plurality of UVC lamps 3.

The casing 2 comprises moreover two ports, respectively an inlet port 4 for supplying air to be sterilized and an outlet port 5 for allowing sterilized air to exit the apparatus.

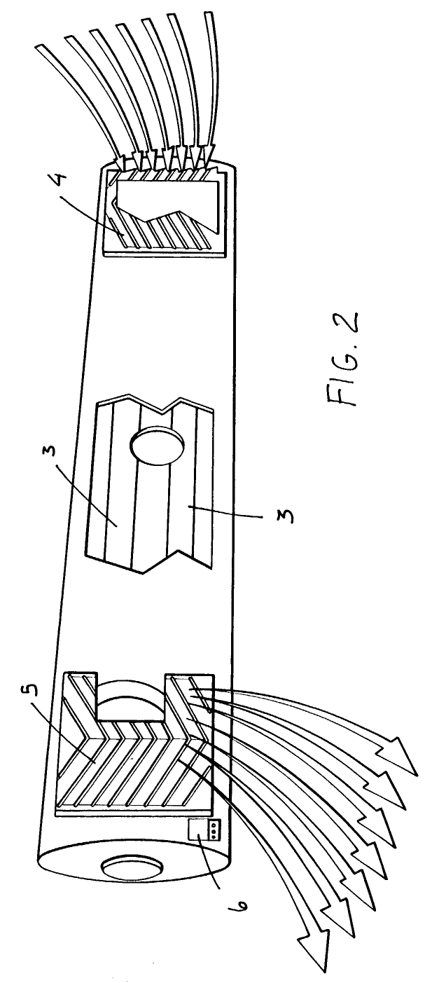

The apparatus 1 comprises furthermore a hour-counter device 6, the portion of which, including a signalling LED system 7 can be seen from the outside of the casing 2 (FIGS. 1 and 2).

The apparatus casing comprises moreover an anti UV glass plate 8, applied on the casing 2 at the height of the lamp 3 and allowing to visually see said lamps from the outside of the apparatus.

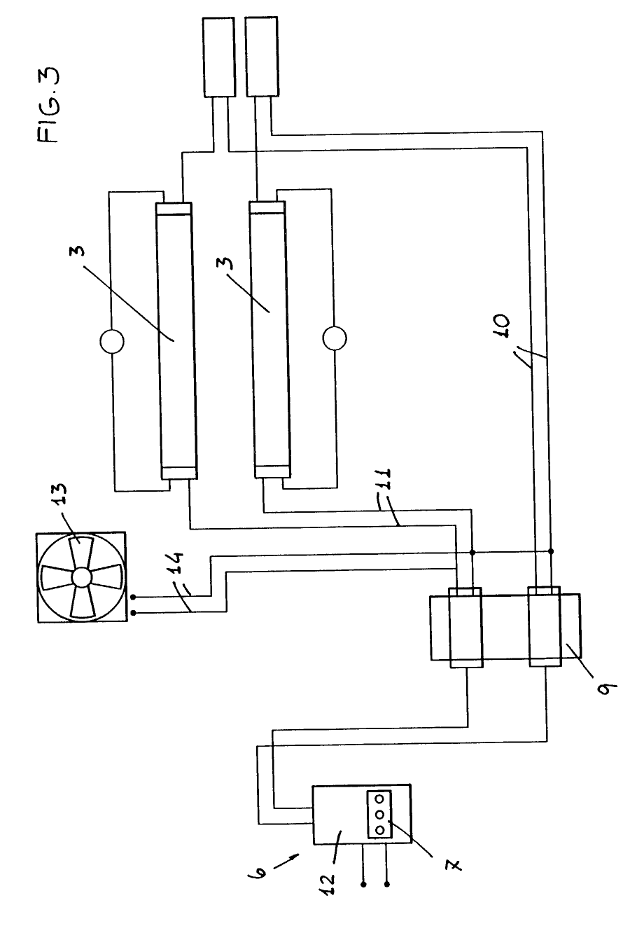

As shown in the circuit diagram of FIG. 3, the lamps 3 and fan 13 are power supplied through the switch-hour counter device 6, through the terminals 9 and respective conductor pairs 10, 11 and 14.

The hour counter device 6 is calibrated on a preset operation hour number of the lamps 3 (usually 6000 hours) with a partial rate of each 2000 hours, indicated by the LED assembly 7.

Advantageously, the device 6 comprises a starting or reset pushbutton or key 12, for resetting the operation of the system after a replacement of exhausted lamps 3.

Before achieving the preset hour limit of operation, the device 6 will indicate timely the incipient exhaustion of the lamps through the last LED, preferably of a red color.

Thus, the user is informed about the incipient exhaustion of the overall apparatus.

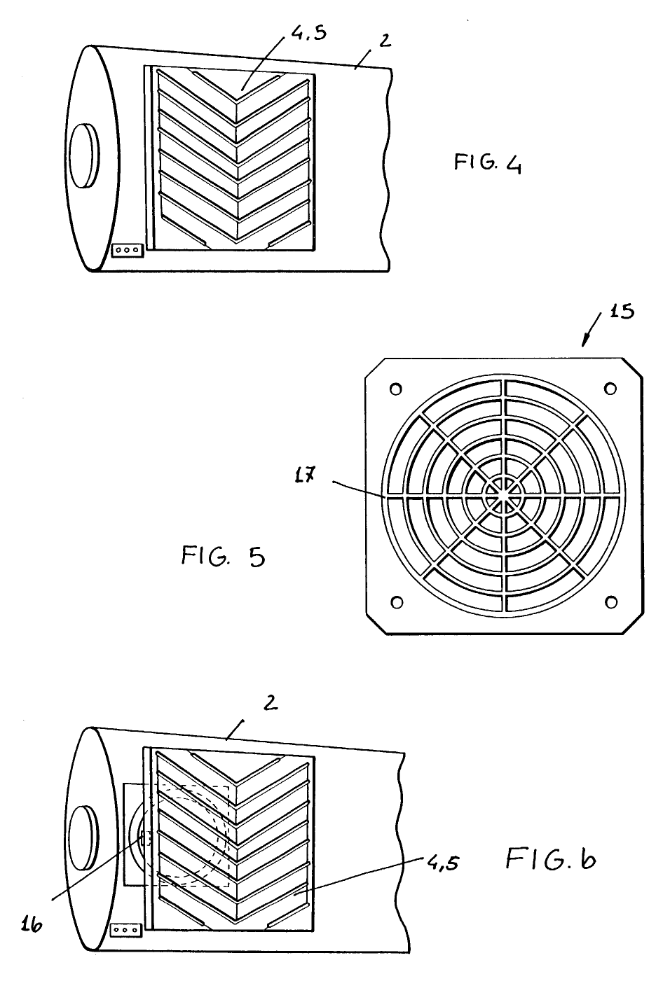

As is shown in FIGS. 4 to 6, the ports 4, 5 are fixed to the casing 2 through the support element 15 shown in FIG. 5, thereon are engaged corresponding snap coupling elements 16, 17 respectively arranged on the inner edge of the ports and related support 15.

Thus, the ports 4, 5 can be applied to the casing 2 according to any desired oriented pattern (see the arrow of FIG. 4).

While the invention has been disclosed with reference to a preferred embodiment thereof, it should be apparent that the disclosed embodiment is susceptible to several modifications and variations all of which will come within the scope of the following claims.

1. A germicide apparatus including UV lamps, characterized in that said apparatus further comprises a device for automatically switching on and off said lamps, before an exhaustion thereof.

2. An apparatus according to claim 1, characterized in that said switching on-off device comprises a switch hour counter device including signalling means for indicating an incipient exhaustion condition of said lamps.

3. An apparatus according to claim 2, characterized in that said signalling means comprise a LED assembly, arranged on a casing of the apparatus.

4. An apparatus according to claim 3, characterized in that said LED's are of a progressive switching-on type being progressively switched on depending on the lamps on time as detected by said hour counter device.

5. An apparatus according to claim 1, characterized in that said apparatus comprises moreover an air circulating fan, said lamps and fan being power supplied through said switch-hour counter device through dedicated terminals and conductor pairs.

6. An apparatus according to claim 1, characterized in that said switch-hour counter device comprises a starting key.

7. An apparatus according to claim 1, characterized in that said casing of said apparatus comprises a first port for allowing air to be sterilized to be supplied therethrough and a second port for allowing sterilized air to exit therethrough, said ports being connected to said casing by a supporting element thereon are engaged snap means respectively located on the inner edge of said ports and on said supporting element.

8. An apparatus according to claim 7, characterized in that said casing comprises an anti-UV glass plate allowing said lamps to be visually seen from the outside of said casing.

9. An apparatus according to claim 1, characterized in that said lamps are uncovered, either of a direct or indirect light type.

10. An apparatus according to claim 1, characterized in that said lamps are UVC lamps.

11. An apparatus according to claim 1, characterized in that said apparatus comprises an ionizing device.

12. An apparatus according to claim 1, characterized in that said switch-hour counter device is so designed as to switch off said lamps after an overall operation time of 6000 hours.
